(19) United States
(12) Patent Application Publication
(10) Pub. No.: US 2007/0238981 A1
(43) Pub. Date: Oct. 11, 2007

(54) Methods and apparatuses for recording and reviewing surgical navigation processes

(75) Inventors: Chuanggui Zhu, Singapore (SG); Kusuma Agusanto, Singapore (SG)

Correspondence Address: GREENBERG TRAURIG, LLP (SV);IP DOCKETING, 2450 COLORADO AVENUE, SUITE 400E, SANTA MONICA, CA, 90404, US

(73) Assignee: Bracco Imaging SPA

(21) Appl. No.: 11/374,684

(22) Filed: Mar. 13, 2006

(51) Int. Cl. A61B 5/05

(52) U.S. Cl. 600/424

(57) ABSTRACT

Methods and apparatuses to record and review a navigation process of image guided surgery. One embodiment includes recording a sequence of positional data to represent a position or position and orientation of a navigation instrument relative to a patient during a surgical navigation process. Another embodiment includes: tracking positions and orientations of a probe during a surgical navigation process; and recording the positions and orientations of the probe, the recording of the positions and orientations to be used to subsequently generate images based on preoperative images of a patient. A further embodiment includes: reading a recorded sequence of locations of a navigational instrument; reading recorded video; generating a sequence of views of three dimensional image data based on the recorded sequence of locations; and combining the sequence of views with corresponding frames of the recorded video.

### TECHNOLOGY FIELD

At least some embodiments of the present invention relate to recording and reviewing of image guided surgical navigation processes in general and, particularly but not exclusively, to recording and reviewing of augmented reality enhanced surgical navigation processes with a video camera.

### BACKGROUND

During a surgical procedure, a surgeon cannot see beyond the exposed surfaces without the help from any visualization equipments. Within the constraint of a limited surgical opening, the exposed visible field may lack the spatial clues to comprehend the surrounding anatomic structures. Visualization facilities may provide the spatial clues which may not be otherwise available to the surgeon and thus allow Minimally Invasive Surgery (MIS) to be performed, dramatically reducing the trauma to the patient.

Many imaging techniques, such as Magnetic Resonance Imaging (MRI), Computed Tomography (CT) and three-dimensional Ultrasonography (3DUS), are currently available to collect volumetric internal images of a patient without a single incision. However, for a number of reasons, such imaging techniques may not be suitable for providing real time images to help a surgeon to comprehend the surgical site during the surgical operation. For example, the processing speed of some of the imaging techniques may not be fast enough to provide real time images with a sufficient resolution; the use of some of the imaging techniques may interfere with the surgical operation; etc.

Further, different techniques for obtaining volumetric, scanned internal images, such as MRI, CT, 3DUS, may be suitable for the visualization of certain structures and tissues but not the others. Thus, these imaging techniques are typically used for diagnosis and planning before a surgical procedure.

Using these scanned images, the complex anatomy structures of a patient can be visualized and examined; critical structures can be identified, segmented and located; and surgical approach can be planned.

The scanned images and surgical plan can be mapped to the actual patient on the operating table and a surgical navigation system can be used to guide the surgeon during the surgery.

U.S. Pat. No. 5,383,454 discloses a system for indicating the position of a tip of a probe within an object on cross-sectional, scanned images of the object. The position of the tip of the probe can be detected and translated to the coordinate system of cross-sectional images. The cross-sectional image closest to the measured position of the tip of the probe can be selected; and a cursor representing the position of the tip of the probe can be displayed on the selected image.

U.S. Pat. No. 6,167,296 describes a system for tracking the position of a pointer in real time by a position tracking system. Scanned image data of a patient is utilized to dynamically display 3-dimensional perspective images in real time of the patient's anatomy from the viewpoint of the pointer.

International Patent Application Publication No. WO 02/100284 A1 discloses a guide system in which a virtual image and a real image are overlaid together to provide visualization of augmented reality. The virtual image is generated by a computer based on CT and/or MRI images which are co-registered and displayed as a multi-modal stereoscopic object and manipulated in a virtual reality environment to identify relevant surgical structures for display as 3D objects. In an example of see through augmented reality, the right and left eye projections of the stereo image generated by the computer are displayed on the right and left LCD screens of a head mounted display. The right and left LCD screens are partially transparent such that the real world seen through the right and left LCD screens of the head mounted display is overlaid with the computer generated stereo image. In an example of microscope assisted augmented reality, the stereoscopic video output of a microscope is combined, through the use of a video mixer, with the stereoscopic, segmented 3D imaging data of the computer for display in a head mounted display. The crop plane used by the computer to generate the virtual image can be coupled to the focus plane of the microscope. Thus, changing the focus value of the microscope can be used to slice through the virtual 3D model to see details at different planes.

International Patent Application Publication No. WO 2005/000139 A1 discloses a surgical navigation imaging system, in which a micro-camera can be provided in a hand-held navigation probe. Real time images of an operative scene from the viewpoint of the micro-camera can be overlaid with computer generated 3D graphics, which depicts structures of interest from the viewpoint of the micro-camera. The computer generated 3D graphics are based on pre-operative scans. Depth perception can be enhanced through varying transparent settings of the camera image and the superimposed 3D graphics. A virtual interface can be displayed adjacent to the combined image to facilitate user interaction.

In at least one embodiment of the present invention, it is desirable to record a surgical navigation process, for reviewing of the surgical process, training, and documentation, etc.

### SUMMARY OF THE DESCRIPTION

Methods and apparatuses to record information and review navigation processes in image sequences with computer generated content are described herein. Some embodiments are summarized in this section.

One embodiment includes recording a sequence of positional data to represent a location of a navigation instrument relative to a patient during a surgical navigation process.

Another embodiment includes: tracking positions and orientations of a probe during a surgical navigation process; and recording the positions and orientations of the probe, the recording of the positions and orientations to be used to subsequently generate images based on preoperative images of a patient.

A further embodiment includes: receiving a location of a camera from a tracking system; recording a frame of video from the camera; and separately recording the location of the camera in association with the frame of the video.

A further embodiment includes: reading a recorded sequence of locations of a navigational instrument; reading recorded video; generating a sequence of views of three dimensional image data based on the recorded sequence of locations; and combining the sequence of views with corresponding frames of the recorded video.

A further embodiment includes: recording video from a camera during a surgical procedure; determining a position and orientation of the camera relative to a subject of the procedure; generating view of three dimensional image data using the determined position and orientation of the camera; and recording positions of the camera during said recording of the video.

One embodiment includes regenerating the navigation process from the recorded data for reviewing the navigation process recorded.

A further embodiment includes regenerating the navigation process the same as what is displayed during the image guided procedure, or with modifications. For example, during the review of the recorded navigation process, the navigation display sequence may be reconstructed to be the same as what is displayed during the image guided procedure, as if the navigation display sequence were recorded as a video stream. Alternatively, the navigation display sequence may be constructed with modifications, such as toggling the visibility of virtual objects, changing transparency, zooming, etc.

A further embodiment includes recording the navigation process as a video image sequence during reviewing. Once recorded as a video image sequence, the video can be played on a variety of machines.

The present invention includes methods and apparatuses which perform these methods, including data processing systems which perform these methods, and computer readable media which when executed on data processing systems cause the systems to perform these methods.

Other features of the present invention will be apparent from the accompanying drawings and from the detailed description which follows.

### BRIEF DESCRIPTION OF THE DRAWINGS

The present invention is illustrated by way of example and not limitation in the figures of the accompanying drawings in which like references indicate similar elements.

FIGS. 1-5 illustrate image recording in an augmented reality visualization system according to one embodiment of the present invention.

FIG. 6 illustrates a method to record and review image sequences according to one embodiment of the present invention.

FIG. 7 illustrates an example of recording sequences according to one embodiment of the present invention.

FIG. 8 shows a flow diagram of a method to record an image guided procedure according to one embodiment of the present invention.

FIG. 9 shows a flow diagram of a method to review a recorded image guided procedure according to one embodiment of the present invention.

FIG. 10 shows a flow diagram of a method to prepare a model in a augmented reality visualization system according to one embodiment of the present invention.

FIG. 11 illustrates a way to generate an image for augmented reality according to one embodiment of the present invention.

FIG. 12 shows a block diagram example of a data processing system for recording and/or reviewing an image guided procedure with augmented reality according to one embodiment of the present invention.

### DETAILED DESCRIPTION

The following description and drawings are illustrative of the invention and are not to be construed as limiting the invention. Numerous specific details are described to provide a thorough understanding of the present invention. However, in certain instances, well known or conventional details are not described in order to avoid obscuring the description. References to one or an embodiment in the present disclosure can be, but not necessarily are, references to the same embodiment; and, such references mean at least one.

According to one embodiment of the present invention, it is desirable to record a surgical navigation process. The recording of the navigation process can be used for reviewing of the surgical process, training, and documentation. In one embodiment, the recording is performed with no or minimal effect on the surgical navigation process.

One embodiment of the present invention provides a system and method to record an augmented reality based image guided navigation procedure. There are many advantages to record information according to embodiments of the present invention. In one embodiment, the position tracking data used to generate the computer images to show the augmented reality and/or to provide image based guidance can be recorded such that, after the procedure, the images provided in the image guided procedure can be recreated for review. The recorded data allows a user to review the procedure with a variety of options. For example, the same images that were displayed in the image guided procedure can be created during the review; and a video clip of what has been shown in the image guided procedure can be created. Alternatively, some of the parameters can be modified to study different aspects of the image guided procedure, which may not be presented during the image guided procedure. In one embodiment, video images captured during the image guided procedure are recorded separately so that, after the procedure, the video images can be reviewed, with or without the augmented content, or with different augmented content. Thus, recording according to embodiments of the present invention allows a variety of flexibilities in reviewing the image guided procedure.

In one example, reality based images that are captured in real time during the procedure are recorded during the surgical navigation process together with related data that is used to construct the augmented reality display in real time during the navigation. Using the recorded data, the augmented reality display sequence can be reconstructed from the recorded images and the recorded data, with or without modification. For example, what is recorded may include at least some of:

1) real time real-world images (e.g., video images from a video camera), which may be recorded in a compressed format or a non-compressed format and which are overlaid with virtual images to generate the augmented reality display during the procedure (e.g., an image guided neurosurgical procedure);

2) plan data used and/or displayed during the procedure to augment reality (e.g., virtual objects, landmarks, measurement, etc., such as tumors, blood vessels, nerves, surgical path, pre-identified anatomical landmarks);

3) rendering parameters (e.g., lighting, color, transparency, visibility, etc.), which can be used in generating the virtual images of the plan data;

4) registration data, which can be used in generating the virtual images and/or overlaying the real-world images and the virtual images;

5) camera properties (e.g., focal length, distortion parameters, etc.), which can be used in generating the virtual images of the virtual objects;

6) the position and orientation of the camera during the procedure, which can be used in generating the virtual images and/or overlaying the real-world images and the virtual images; and

7) synchronizing information to correlate sequences of recorded data.

The recorded data can be used to rebuild an augmented reality display sequence. For example, a method to rebuild a display sequence may include at least some of:

1) retrieving the recorded real-world images;

2) regenerating the virtual images according to the recorded data;

3) synchronizing the virtual images and real-word images; and

4) combining the virtual images and video images to show an augmented reality display sequence.

When the display sequence is rebuilt, the augmented reality display sequence can be recorded as a video image sequence to reduce memory required to store the display sequence and to reduce the processing required to playback the same display sequence. Once recorded as a video image sequence, the video can be played on a variety of machines.

In one embodiment, the regenerated augmented reality display sequence may be substantially the same as what is displayed during the image guided procedure, or with modifications. For example, during the review of an image guided procedure, the augmented reality display sequence may be reconstructed to be the same as what is displayed during the image guided procedure, as if the augmented reality display sequence were recorded as a video stream. Alternatively, the augmented reality display sequence may be constructed with modifications, such as toggling the visibility of virtual objects, changing transparency, zooming, etc. Further, the virtual image sequences and the real-world image sequences may be viewed separately.

The data for the generation of the virtual images may be modified during a review process. For example, rendering parameters may be adjusted during the review process, with or without pausing the playing back of the sequence. For example, new, updated virtual objects may be used to generate a new augmented reality display sequence using the recorded reality based image sequence.

One embodiment of the present invention arranges to transmit the information for an image guided procedure through a network connection to a remote site for reviewing or monitoring without affecting the performance of the real time display for the image guided procedure. Example details on a system to display over a network connection may be found in Provisional U.S. Patent Application No. 60/755,658, filed Dec. 31, 2005 and entitled “Systems and Method for Collaborative Interactive Visualization Over a Network”, which is hereby incorporated herein by reference.

For example, the speed of the video of the image guided procedure may be adjusted so that the display sequence may be transmitted using the available bandwidth of a network to a remote location for review. For example, the frame rate may be decreased to stream the image guided procedure at a speed slower than the real time display in the surgical room, based on the availability of the network bandwidth. Alternatively, the frame rate may be decreased (e.g., through selectively dropping frames) to stream the image guided procedure at the same speed as the real time display in the surgical room, based on the availability of the network bandwidth.

For example, the recorded data can be sent to a remote location when it is determined that the system is idle or has enough resources. Thus, the transmission of the data for the display of the image guided procedure for monitoring and reviewing at a remote site may be performed asynchronously with the real time display of the image guided procedure. The remote site may reconstruct the display of the image guided procedure with a time shift (e.g., with a delay from real time to have an opportunity to review or monitor a portion of the procedure while the procedure is still in progress).

In one embodiment of the present invention, the recording of the image guided procedure may further include the recording of information that can be used to code the recorded sequence so that the sequence can be easily searched, organized and linked with other resources. For example, the sequence may be recorded with tags applied during the image guided procedure. The tags may include one or more of: time, user input/interactions (e.g., text input, voice input, text recognized from voice input, markings provided through a graphical user interface), user interaction events (e.g., user selection of an virtual object, zoom change, application of tags defined during the planning prior to the image guided procedure), etc.

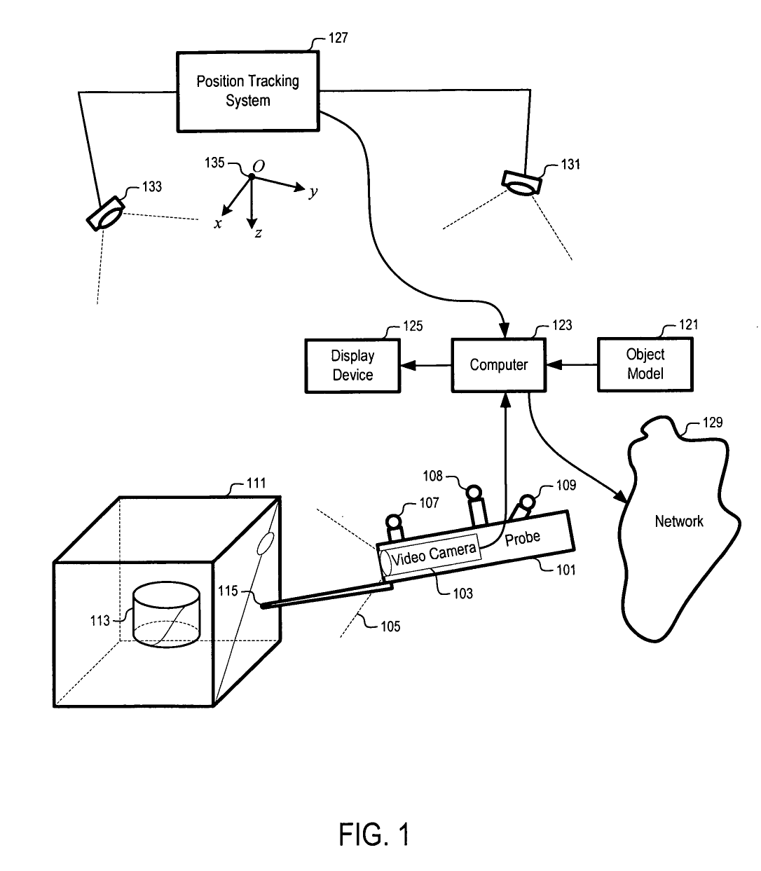

FIGS. 1-5 illustrate image recording in an augmented reality visualization system according to one embodiment of the present invention. In FIG. 1, a computer (123) is used to generate a virtual image of a view, according to a viewpoint of the video camera (103), to enhance the display of the reality based image captured by the video camera (103). The reality image and the virtual image are mixed in real time for display on the display device (125) (e.g., a monitor, or other display devices). The computer (123) generates the virtual image based on the object model (121) which is typically generated from scan images of the patient and defined before the image guided procedure (e.g., a neurosurgical procedure).

In FIG. 1, the video camera (103) is mounted on a probe (101) such that a portion of the probe, including the tip (115), is in the field of view (105) of the camera. The video camera (103) may have a known position and orientation with respect to the probe (101) such that the position and orientation of the video camera (103) can be determined from the position and the orientation of the probe (101).

In FIG. 1, the position and the orientation of the probe (101) relative to the object of interest (111) may be changed during the image guided procedure. The probe (101) may be hand carried and positioned to obtain a desired view.

In FIG. 1, the position and orientation of the probe (101), and thus the position and orientation of the video camera (103), is tracked using a position tracking system (127).

For example, the position tracking system (127) may use two tracking cameras (131 and 133) to capture the scene in which the probe (101) is. The probe (101) has features (107, 108 and 109) (e.g., tracking balls). The image of the features (107, 108 and 109) in images captured by the tracking cameras (131 and 133) can be automatically identified using the position tracking system (127). Based on the positions of the features (107, 108 and 109) of the probe (101) in the video images of the tracking cameras (131 and 133), the position tracking system (127) can compute the position and orientation of the probe (101) in the coordinate system (135) of the position tracking system (127).

The image data of a patient, including the various objects associated with the surgical plan which are in the same coordinate systems as the image data, can be mapped to the patient on the operating table using one of the generally known registration techniques. For example, one such registration technique maps the image data of a patient to the patient using a number of anatomical features (at least 3) on the body surface of the patient by matching their positions identified and located in the scan images and their corresponding positions on the patient determined using a tracked probe. The registration accuracy may be further improved by mapping a surface of a body part of the patient generated from the imaging data to the surface data of the corresponding body part generated on the operating table. Example details on registration may be found in U.S. patent application Ser. No. 10/480,715, filed Jul. 21, 2004 and entitled “Guide System and a Probe Therefor”, which is hereby incorporated herein by reference.

A reference frame with a number of fiducial points marked with markers or tracking balls can be attached rigidly to the interested body part of the patient so that the position tracking system (127) may also determine the position and orientation of the patient even if the patient is moved during the surgery.

The position and orientation of the object (e.g. patient) (111) and the position and orientation of the video camera (103) in the same reference system can be used to determine the relative position and orientation between the object (111) and the video camera (103). Thus, using the position tracking system (127), the viewpoint of the camera with respect to the object (111) can be tracked.

Although FIG. 1 illustrates an example of using tracking cameras in the position tracking system, other types of position tracking systems may also be used. For example, the position tracking system may determine a position based on the delay in the propagation of a signal, such as a radio signal, an ultrasound signal, or a laser beam. A number of transmitters and/or receivers may be used to determine the propagation delays to a set of points to track the position of a transmitter (or a receiver). Alternatively, or in combination, for example, the position tracking system may determine a position based on the positions of components of a supporting structure that may be used to support the probe.

Further, the position and orientation of the video camera (103) may be adjustable relative to the probe (101). The position of the video camera relative to the probe may be measured (e.g., automatically) in real time to determine the position and orientation of the video camera (103).

Further, the video camera may not be mounted in the probe. For example, the video camera may be a separate device which may be tracked separately. For example, the video camera may be part of a microscope. For example, the video camera may be mounted on a head mounted display device to capture the images as seen by the eyes through the head mounted display device. For example, the video camera may be integrated with an endoscopic unit.

Further, other types of real time imaging devices may also be used, such as ultrasonography.

During the image guided procedure, the position and/or orientation of the video camera (103) relative to the object of interest (111) may be changed. A position tracking system is used to determine the relative position and/or orientation between the video camera (103) and the object (111).

The object (111) may have certain internal features (e.g., 113) which may not be visible in the video images captured using the video camera (103). To augment the reality based images captured by the video camera (103), the computer (123) may generate a virtual image of the object based on the object model (121) and combine the reality based images with the virtual image.

In one embodiment, the position and orientation of the object (111) correspond to the position and orientation of the corresponding object model after registration. Thus, the tracked viewpoint of the camera can be used to determine the viewpoint of a corresponding virtual camera to render a virtual image of the object model (121). The virtual image and the video image can be combined to display an augmented reality image on display device (125).

In one embodiment of the present invention, instead of recording what is displayed on the display device (125), the data used by the computer (123) to generate the display on the display device (125) is recorded such that it is possible to regenerate what is displayed on the display device (125), to generate a modified version of what is displayed on the display device (125), to transmit data over a network (129) to reconstruct what is displayed on the display device (125) while avoiding affecting the real time processing for the image guided procedure (e.g., transmit with a time shift during the procedure, transmit in real time when the resource permits, or transmit after the procedure).

The 3D model may be generated from three-dimensional (3D) images of the object (e.g., bodies or body parts of a patient). For example, a MRI scan or a CAT (Computer Axial Tomography) scan of a head of a patient can be used in a computer to generate a 3D virtual model of the head.

Different views of the virtual model can be generated using a computer. For example, the 3D virtual model of the head may be rotated seemly in the computer so that another point of view of the model of the head can be viewed; parts of the model may be removed so that other parts become visible; certain parts of the model of the head may be highlighted for improved visibility; an interested area, such as a target anatomic structure, may be segmented and highlighted; and annotations and markers such as points, lines, contours, texts, labels can be added to into the virtual model.

In a scenario of surgical planning, the viewpoint is fixed, supposedly corresponding to the position(s) of the eye(s) of the user; and the virtual model is movable in response to the user input. In a navigation process, the virtual model is registered to the patient and is generally still. The camera can be moved around the patient; and a virtual camera, which may have the same viewpoint, focus length, field of view etc, position and orientation as of the real camera, is moved according to the movement of the real camera. Thus, different views of the object is rendered from different viewpoints of the camera.

Viewing and interacting virtual models generated from scanned data can be used for planning the surgical operation. For example, a surgeon may use the virtual model to diagnose the nature and extent of the medical problems of the patient, and to plan the point and direction of entry into the head of the patient for the removal of a tumor to minimize damage to surrounding structure, to plan a surgical path, etc. Thus, the model of the head may further include diagnosis information (e.g., tumor object, blood vessel object), surgical plan (e.g., surgical path), identified landmarks, annotations and markers. The model can be generated to enhance the viewing experience and highlight relevant features.

During surgery, the 3D virtual model of the head can be used to enhance reality based images captured from a real time imaging device for surgery navigation and guidance. For example, the 3D model generated based on preoperatively obtained 3D images produced from MRI and CAT (Computer Axial Tomography) scanning can be used to generate a virtual image as seen by a virtual camera. The virtual image can be superimposed with an actual surgical field (e.g., a real-world perceptible human body in a given 3D physical space) to augmented reality (e.g., see through a partially transparent head mounted display), or mixed with a video image from a video camera to generate an augmented reality display. The video images can be captured to represent the reality as seen. The video images can be recorded together with parameters used to generate the virtual image so that the reality may be reviewed later without the computer generated content, or with a different computer generated content, or with the same computer generated content.

Thus, the reality as seen through the partially transparent head mounted display may be captured and used. The viewpoint of the head mounted display can be tracked and recorded so that the display provided in the partially transparent head mounted display can be reconstructed for review after the procedure, with or without modification. Based on the reconstruction of the display provided in the partially transparent head mounted display, a video of what is displayed during the procedure can be regenerated, reviewed and recorded after the procedure.

Further, the probe (101) may not have a video camera mounted within it. The real time position and orientation of the probe (101) relative to the object (111) can be tracked using the position tracking system (127). A viewpoint associated with the probe (101) can be determined to construct a virtual view of the object model (121), as if a virtual camera were at the viewpoint associated with the probe (101). The computer (123) may generate a real time sequence of images of the virtual view of the object model (121) for display on the display device to guide the navigation of the probe (101), with or without the real time video images from a video camera mounted in the probe. In one embodiment, the probe does not contain a micro video camera; and the probe can be represented by an icon that is displayed on the virtual view of the object model, or displayed on cross-sectional views of a scanned 3D image set, according to the tracked position and orientation of the probe.

Image based guidance can be provided based on the real time position and orientation relation between the object (111) and the probe (101) and the object model (121). Based on the known geometric relation between the viewpoint and the probe (101), the computer may further generate a representation of the probe (e.g., using a 3D model of the probe) to show the relative position of the probe with respect to the object.

For example, the computer (123) can generate a 3D model of the real time scene having the probe (101) and the object (111), using the real time determined position and orientation relation between the object (111) and the probe (101), a 3D model of the object (111), and a model of the probe (101). With the 3D model of the scene, the computer (123) can generate a view of the 3D model of the real time scene from any viewpoint specified by the user. Thus, the viewpoint for generating the display on the display device may be a viewpoint with a pre-determined geometric relation with the probe (101) or a viewpoint as specified by the user in real time during the image guided procedure. Alternatively, the probe may be represented using an icon.

In one embodiment, information indicating the real time position and orientation relation between the object (111) and the probe (101) and the real time viewpoint for the generation of the real time display of the image for guiding the navigation of the probe is recorded so that, after the procedure, the navigation of the probe may be review from the same sequence of viewpoints, or from different viewpoints, with or without any modifications to the 3D model of the object (111) and the model of the probe (101).

Note that various medical devices, such as endoscopes, can be used as a probe in the navigation process.

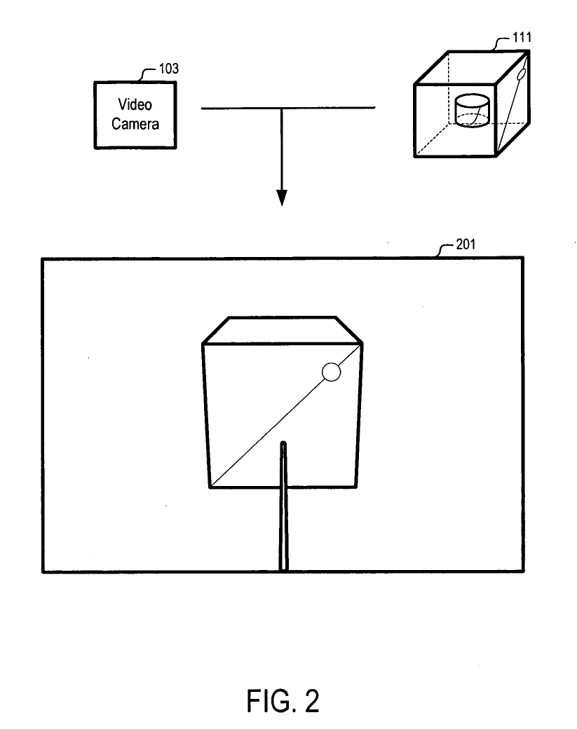

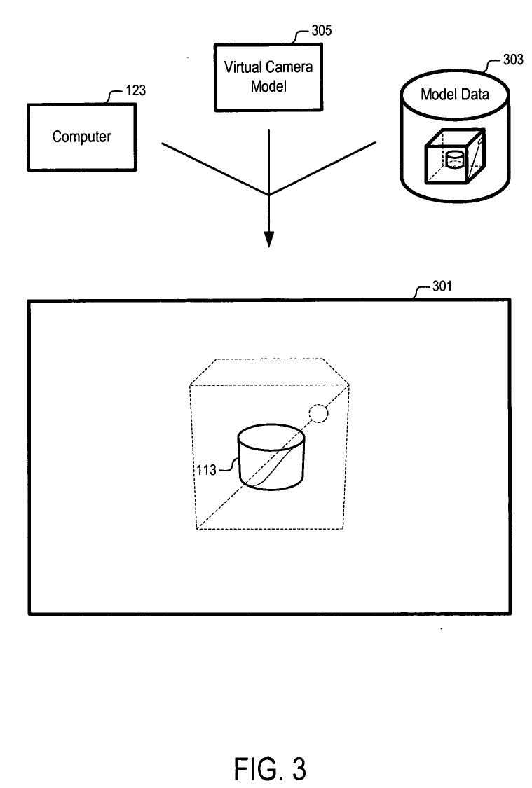

In FIG. 2, a video camera (103) captures a frame of a video image (201) which shows on the surface features of the object (111) from a view point that is tracked. In FIG. 3, a computer (123) uses the model data (303), which may be a 3D virtual reality model of the object (e.g., generated based on volumetric imaging data, such as MRI or CT scan), and the virtual camera model (305) to generate the virtual image (301) as seen by a virtual camera. The sizes of the images (201 and 301) may be the same.

In one embodiment, the virtual camera is defined to have the same viewpoint as the video camera such that the virtual camera has the same viewing angle and/or viewing distance to the 3D model of the object as the video camera to the real object. The computer (123) selectively renders the internal feature (113) (e.g., according to a user request). For example, the 3D model may contain a number of user selectable objects; and one or more of the objects may be selected to be visible based on a user input or a pre-defined selection criterion (e.g., based on the position of the focus plane of the video camera).

The virtual camera may have a focus plane defined according to the video camera such that the focus plane of the virtual camera corresponding to the same focus plane of the video camera, relative to the object. Alternatively, the virtual camera may have a focus plane that is a pre-determined distance further away from the focus plane of the video camera, relative the object.

The virtual camera model may include a number of camera parameters, such as field of view, focal length, distortion parameters, etc. The generation of virtual image may further include a number of rendering parameters, such as lighting condition, color, and transparency. Some of the rendering parameters may correspond to the settings in the real world (e.g., according to the real time measurements), some of the rendering parameters may be pre-determined (e.g., pre-selected by the user), some of the rendering parameters may be adjusted in real time according to the real time user input.

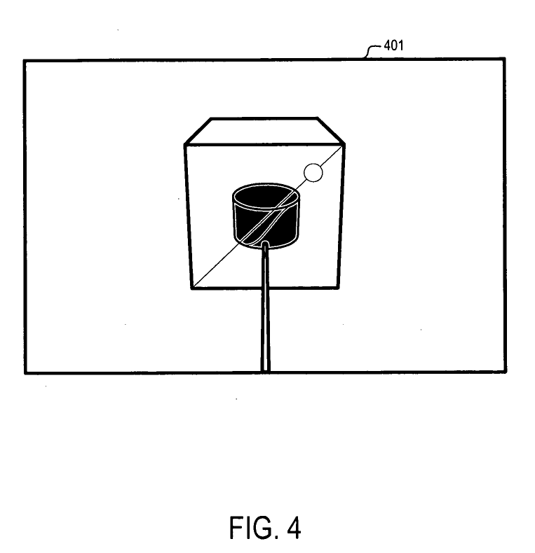

The video image (201) in FIG. 2 and the computer generated image (301) in FIG. 3, as captured by the virtual camera, can be combined to show the image (401) of augmented reality in real time in FIG. 4.

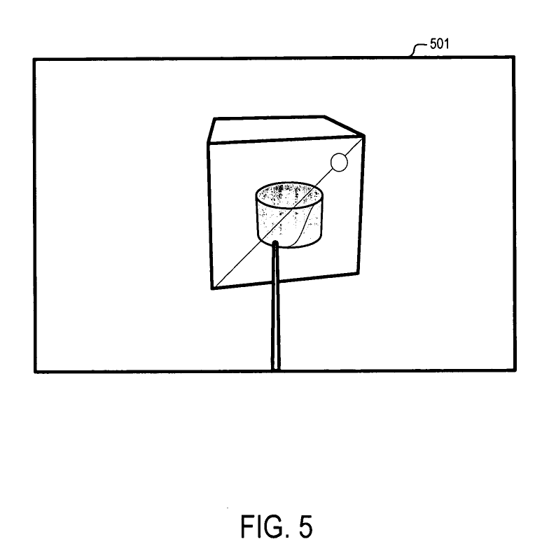

When the position and/or the orientation of the video camera (103) is changed, the image captured by the virtual camera is also changed; and the combined image (501) of augmented reality is also changed, as shown in FIG. 5.

In one embodiment, the information used by the computer to generate the image (301) is recorded, separately from the video image (201), so that the video image (201) may be reviewed without the computer generated image (301) (or with a different computer generated image).

In one embodiment, the video image (201) may not be displayed to the user for the image guided procedure. The video image (201) may correspond to a real world view seen by the user through a partially transparent display of the computer generated image (301); and the video image (201) is captured so that what is seen by the user may be reconstructed on a display device after the image guided procedure, or on a separate device during the image guided procedure for monitoring.

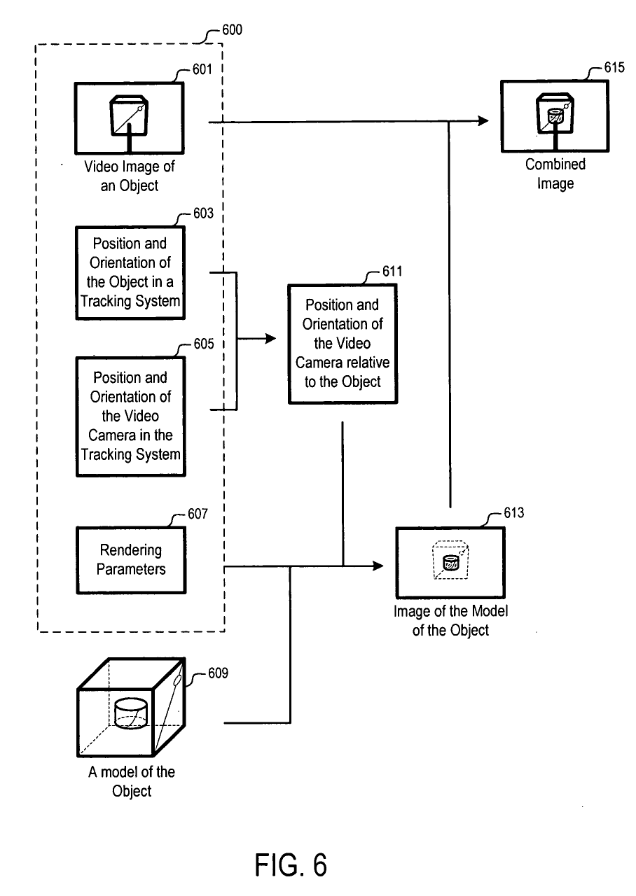

FIG. 6 illustrates a method to record and review image sequences according to one embodiment of the present invention. In FIG. 6, a model of the object (609) is generated using the volumetric images obtained prior to the image guided procedure. The model of the object (609) is accessible after the image guided procedure. Further, the model of the object (609) may be updated after the image guided procedure; alternatively, a different model of the object (609) (e.g., based on volumetric images obtained after the image guided procedure) may be used after the image guided procedure.

In FIG. 6, information (600) is recorded for the possibility of reconstruction the real time display of augmented reality. Information (600) includes the video image (601) of an object, the position and orientation (603) of the object in the tracking system, the position and orientation (605) of the video camera in the tracking system, and the rendering parameters (607), which are recorded as a function of a synchronization parameter (e.g., time, frame number) so that for each frame of the video image, the position and orientation (611) of the video camera relative to the object can be determined and used to generate the corresponding image (613) of the model of the object. The image (613) of the model of the object can be combined with the corresponding video image to generate the combined image (615).

In one embodiment, the system records the position and orientation of the video camera relative to the object (611) such that the position and orientation relative to the tracking system may be ignored.

In one embodiment, some of the rendering parameters may be adjusted during the reconstruction, to provide a modified view of the augmented reality.

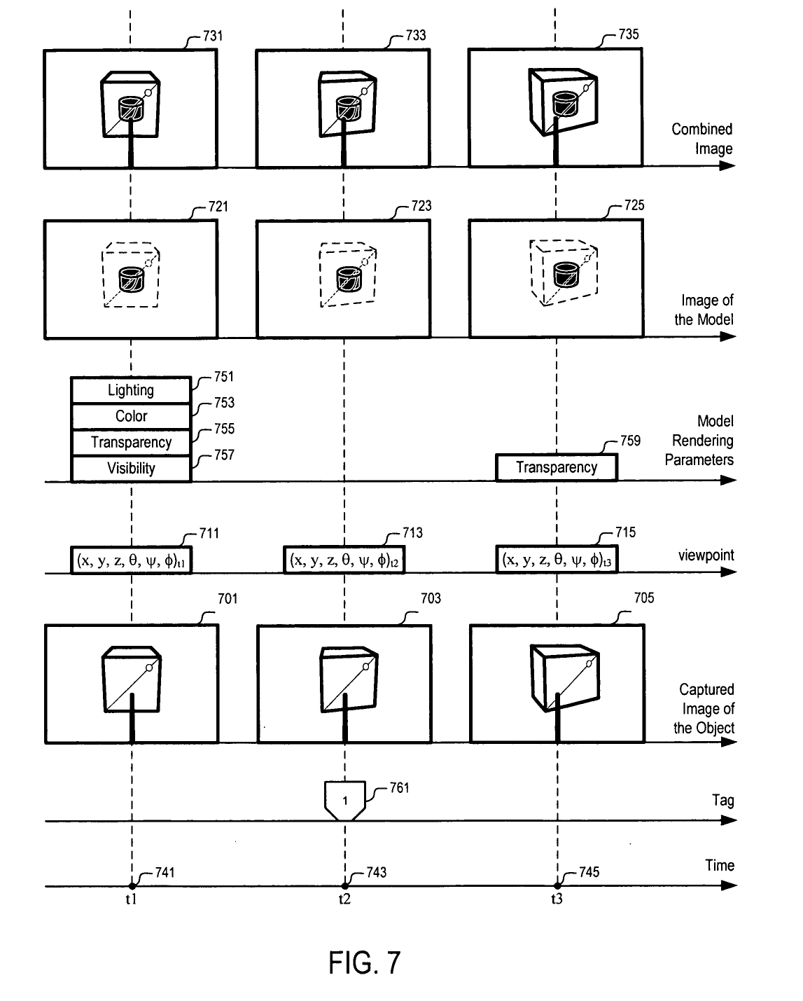

FIG. 7 illustrates an example of recording sequences according to one embodiment of the present invention. In FIG. 7, captured image of the object is recorded (e.g., at a rate of more than ten frames per second, such as 20-25 frames per second). The video images (e.g., 701, 703, 705) may be captured and stored in a compressed format (e.g., a lossy format or a lossless format), or a non-compressed format. During the image guided procedure, the view point of the camera is tracked such that the view points (711, 713, 715) at the corresponding times (741, 743 and 745) at which the video images (701, 703, 705) are captured can be determined and used to generate the images (721, 723 and 725) of the model. The captured images (701, 703, 705) of the object and the images (721, 723, and 725) of the model can be combined to provide combined images (731, 733, 735) to guide the procedure. The recording of the combined images (731, 733, 735) and the images (721, 723, 725) of the model is optional, since these images can be reconstructed from other recorded information.

In one embodiment, information to determine the viewpoint is recorded for each frame of the captured image of the object. Alternatively, the information to determine the viewpoint may be recorded for the corresponding frame when changes in the viewpoint occurs. The system may record the viewpoint of the camera with respect to the object, or other information can be used to derive the viewpoint of the camera with respect to the object, such as the position and orientation of the camera and/or the object in a position tracking system.

In one embodiment, the rendering parameters, such as lighting (751), color (753), transparency (755), visibility (757), etc., are recorded at the time the change to the corresponding parameter (e.g., 759) occurs. Thus, based on the recorded information about the rendering parameters, the rendering parameters used to render each of the images (721, 723, 725) of the model can be determined. Alternatively, a complete set of rendering parameters may be recorded for each frame of the captured image of the object.

In one embodiment, the recording further includes the recording of tag, such as information (761), which can be used to identify a particular portion of the recorded sequence. The tag information may be a predefined indicator correlated with the time or frame of the captured image of the object. The tag information may indicate a particular virtual object of the model entering into or existing from the image sequence of the model (e.g., when the visibility of the virtual object is toggled, such as changing from visible to invisible or changing from invisible to visible). The tag information may include a text message, which may be pre-defined and applied in real time, or typed during the image guided procedure and applied, or recognized from a voice comment during the image guided procedure and applied. The tag information may indicate the starting or ending of a related recording, such as the measurement of a medical equipment. The tag may include a link to a related recorded. The tag information may be used to code the image sequence so that different portions of the sequence can be searched for easy access. In one embodiment, the tag information is recorded at the head of each position and orientation of the probe.

With the recorded information, the combined images and the images of the model for the corresponding captured image of the object can be reconstructed and displayed. Further, some of the parameters, such as the model rendering parameters, the model of the objects may be modified during a review (or prior to the review). Further, additional virtual objects may be added to augment the captured, reality based image (e.g., based on a post-surgery scan of the patient to compare the planning, the surgery, and the result of the surgery).

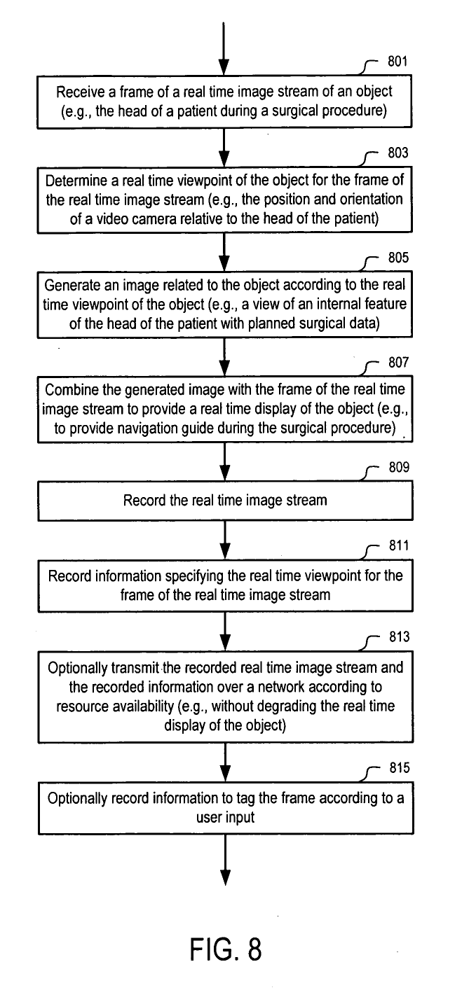

FIG. 8 shows a flow diagram of a method to record an image guided procedure according to one embodiment of the present invention.

In FIG. 8, a frame of a real time image stream of an object (e.g., the head of a patient during a surgical procedure) is received (801) (e.g., to provide guide and/or for recording). A real time viewpoint of the object for the frame of the real time image stream is determined (803) (e.g., the position and orientation of a video camera relative to the head of the patient) to generate (805) an image related to the object according to the real time viewpoint of the object (e.g., a view of an internal feature of the head of the patient with planned surgical data). The image may show features which may not exist in the object in real world, such as a planned surgical path, diagnosis information, etc. The image may show features which may exist in the object in real world, not visible in the real time image stream, such as internal structures, such as a tumor, a blood vessel, a nerve, an anatomical landmark, etc.

The generated image is combined (807) with the frame of the real time image stream to provide a real time display of the object (e.g., to provide navigation guide during the surgical procedure). The real time display of the object is based on augmented reality. Further, user interface elements can also be displayed to allow the manipulation of the display of the augmented reality. For example, the transparent parameter for mixing the real time image stream and the generated image may be adjusted in real time; the user may adjust zoom parameters, toggle the visibility of different virtual objects, apply tags, adjust the focal plane of the virtual camera, make measurements, record positions, comments, etc.

The real time image stream is recorded (809); and the information specifying the real time viewpoint for the frame of the real time image stream is also recorded (811). The recorded image stream and information can be used to reconstruct the display of the object with combined images, with or without modifications. The information specifying the real time viewpoint for the frame of the real time image stream may be tracking data, including one or more of: the data received from the position tracking system, the position and/or orientation of a device (e.g., a video camera or a probe) relative to the object, the orientation of the device relative to the object, the distance from the device to the object, and the position and/or orientation of a virtual camera relative to the 3D model related to the object.

Optionally, the recorded real time image stream and the recorded information can be transmitted (813) over a network according to resource availability (e.g., without degrading the real time display of the object).

Optionally, information to tag the frame can be recorded (815) according to a user input. The information may include indications of events during the recording time period and inputs provided by the user.

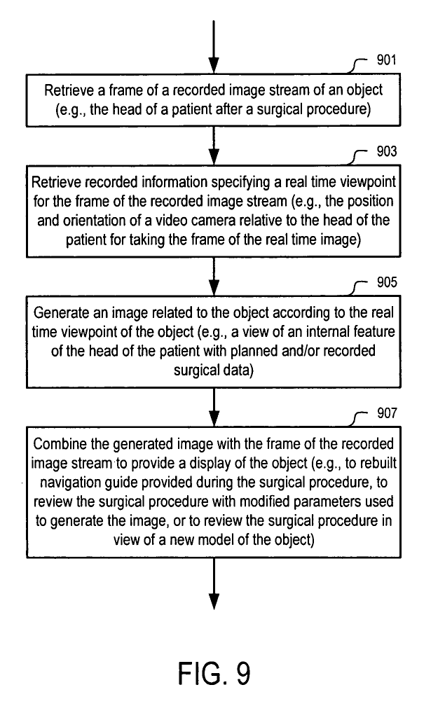

FIG. 9 shows a flow diagram of a method to review a recorded image guided procedure according to one embodiment of the present invention. In FIG. 9, a frame of a recorded image stream of an object (e.g., the head of a patient after a surgical procedure) is retrieved (e.g., for reviewing or for rebuilding a display with augmented reality).

Recorded information specifying a real time viewpoint is retrieved (903) for the frame of the recorded image stream (e.g., the position and orientation of a video camera relative to the head of the patient for taking the frame of the real time image) to generate (905) an image related to the object according to the real time viewpoint of the object (e.g., a view of an internal feature of the head of the patient with planned and/or recorded surgical data).

The generated image is combined (907) with the frame of the recorded image stream to provide a display of the object. For example, the combined image may be generated to rebuilt navigation guide provided during the surgical procedure, to review the surgical procedure with modified parameters used to generate the image, or to review the surgical procedure in view of a new model of the object.

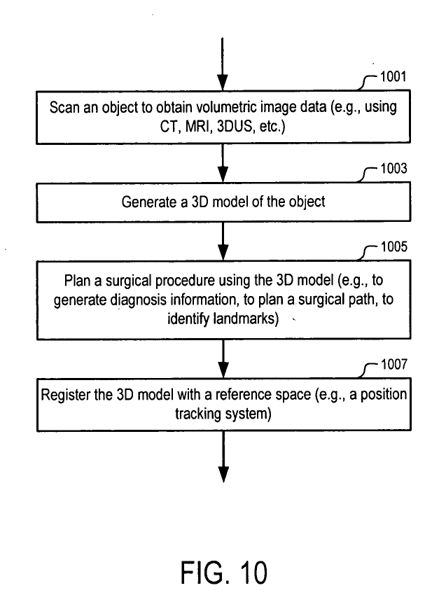

FIG. 10 shows a flow diagram of a method to prepare a model in an augmented reality visualization system according to one embodiment of the present invention. In FIG. 10, an object is scanned (1001) to obtain volumetric image data (e.g., using CT, MRI, 3DUS, etc.), which can be used to generate (1003) a 3D model of the object and plan (1005) a surgical procedure using the 3D model (e.g., to generate diagnosis information, to plan a surgical path, to identify anatomical landmarks). The 3D model is registered with the object.

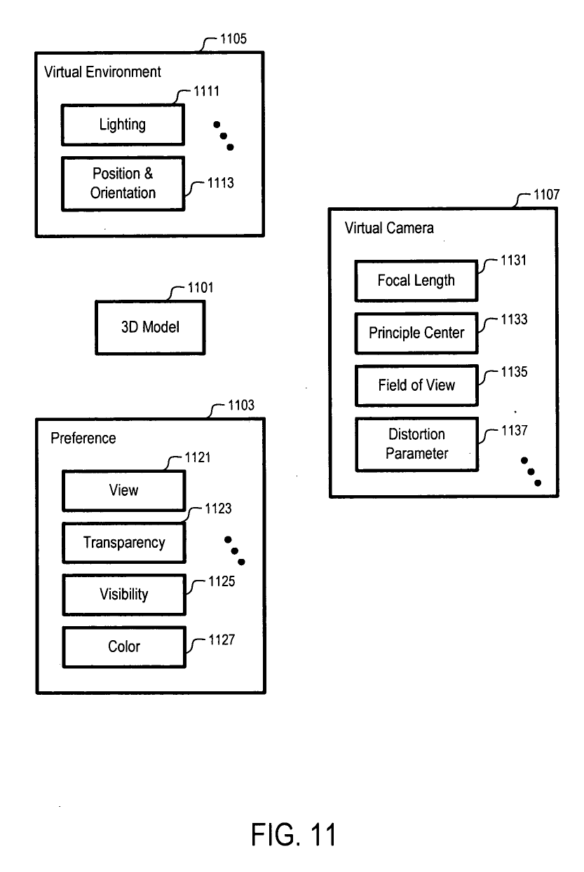

FIG. 11 illustrates a way to generate an image for augmented reality according to one embodiment of the present invention. In FIG. 11, the 3D model (1101) of the object may be the same as the one used during the image guided procedure, or a modified one, or a different one (e.g., generated based on a volumetric image scan after the image guided procedure). The 3D model (1101) is placed in a virtual environment (1105) with lighting (1111) and position and orientation (1113) relative to the light sources and/or other virtual objects (e.g., surgical path, diagnosis information, etc.).

In FIG. 11, a virtual camera (1107) is used to capture an image of the 3D model in the virtual environment (1105). The virtual camera may include parameters such as focal length (1131), principle center (the viewpoint) (1133), field of view (1135), distortion parameter (1137), etc.

The rendering of the image as captured by the virtual camera may further incldue a number of preferences, such as a particular view of the 3D model (e.g., a cross-sectional view, a view with cutout, a surface view, etc.), the transparency (1123) for combining with the recorded video image, the visibility (1125) of different virtual objects, color (1127) of an virtual object, etc.

In one embodiment, some or all of the parameters are based on recorded information. Some of the parameters may be changed for the review.

A surgical navigation process typically includes the controlled movement of a navigation instrument with respect to a patient during a surgical operation. The navigation instrument may be a probe, a surgical instrument, a head mounted display, an imaging device such as a video camera or an ultrasound probe, an endoscope, a microscope, or a combination of such devices. For example, a probe may contain a micro video camera.

During the navigation, images may be displayed in real time to assist navigators in locating locations within the body (or on the body), and position the navigation instrument to a desired location relative to the body. The images displayed may be intraoperative images obtained from imaging devices such as ultrasonography, MRI, X-ray, etc. In one embodiment, images used in navigation, obtained pre-operatively or intraoperatively, can be the images of internal anatomies. To show a navigation instrument inside a body part of a patient, its position can be indicated in the images of the body part. For example, the system can: 1) determine and transform the position of the navigation instrument into the image coordinate system, and 2) register the images with the body part. Using intraoperative images, the images are typically registered with the patient naturally. The system determines the imaging device pose (position and orientation) (e.g., by using a tracking system) to transform the probe position to the image coordinate system.

When no intraoperative image is used, the location of the navigation instrument can be tracked to show the location of the instrument with respect to the subject of the surgical operation. For example, a representation of the navigation instrument, such as an icon, a pointer, a rendered image of a 3D model of the probe, etc., can be overlaid on images obtained before the surgery (preoperative images) to help positioning the navigation instrument relative to the patient. For example, a 3D model of the patient may be generated from the preoperative images; and an image of the navigation instrument can be rendered with an image of the patient, according to the tracked location of the navigation instrument.

When intraoperative images are used, the intraoperative images may capture a portion of the navigation instrument. A representation of the navigation instrument can be overlaid with the intraoperative images, in a way similar to overlaying a representation of the navigation instrument over the preoperative images.

In one embodiment, the imaging devices to collect internal images, such as CT, MRI, ultrasound images, are typically not part of the navigation instrument. However, some imaging devices, such as camera, endoscopes, microscope and ultrasound probe, can be part of the navigation instrument. The imaging device as part of a navigation instrument can have a position determined by a tracking system relative to the images of internal anatomy.

A navigation instrument may have an imaging device. When the imaging device has a pre-determined spatial relation with respect to the navigation instrument, the position and orientation of the tracked navigation instrument can be used to determine the position and orientation of the imaging device. Alternatively, the position and orientation of the imaging device can be tracked separately, or tracked relative to the tracked navigation instrument. The tracking of the imaging device and the tracking of the navigation instrument may be performed using a same position tracking system.

In one embodiment, positional data to represent a position and orientation of a navigation instrument with respect to a patient during a surgical navigation process is recorded. Using the recorded positional data, images of preoperative data can be generated to assist the navigator during surgery, and/or to reconstruct or review the recorded navigation process.

Positional data, as referred herein, may generally refer to data that describes positional relations. It is understood that a positional relation may be represented in many different forms. For example, the positional relation between a navigation instrument and a patient (subject of navigation) may include the relative position and/or orientation between the navigation instrument and the patient. In this description, the term “location” may refer to position and/or orientation.

The relative position and/or orientation between the navigation instrument and the patient may be represented using: a) the position of one representative point of the navigation instrument, and b) the orientation of the navigation instrument, in a coordinate system that is based on the position and orientation of the patient (patient coordinate system). Alternatively, the position and/or orientation of the navigation instrument may be replaced with other data from which the position and orientation of the navigation instrument can be calculated in the patient coordinate system.

It is understood that when the navigation instrument is considered as a rigid body, the position and orientation of the navigation instrument determines the position of any points on the navigation instrument, as well as the position and orientation of any parts of the navigation instrument.

Similarly, when the navigation instrument is considered as a rigid body, the positions of a number of points of the navigation instrument can determine the orientation of the navigation instrument.

Further, the position of the representative point of the navigation instrument can be replaced with: a) the orientation angles of the representative point with respect to the patient coordinate system, and b) the distance between the representative point and the origin of the patient coordinate system. Furthermore, it is not necessary to describe the relative position and/or orientation between the navigation instrument and the patient in the patient coordinate system. For example, the position and orientation between the navigation instrument and the patient can be represented using the position and orientation of the navigation instrument in a position tracking system and the position and orientation of the patient in the position tracking system.

Thus, positional data to represent a positional relation is not limited to a specific form. Some forms of positional data are used as examples to describe the positional relations. However, it is understood that positional data are not limited to the specific examples.

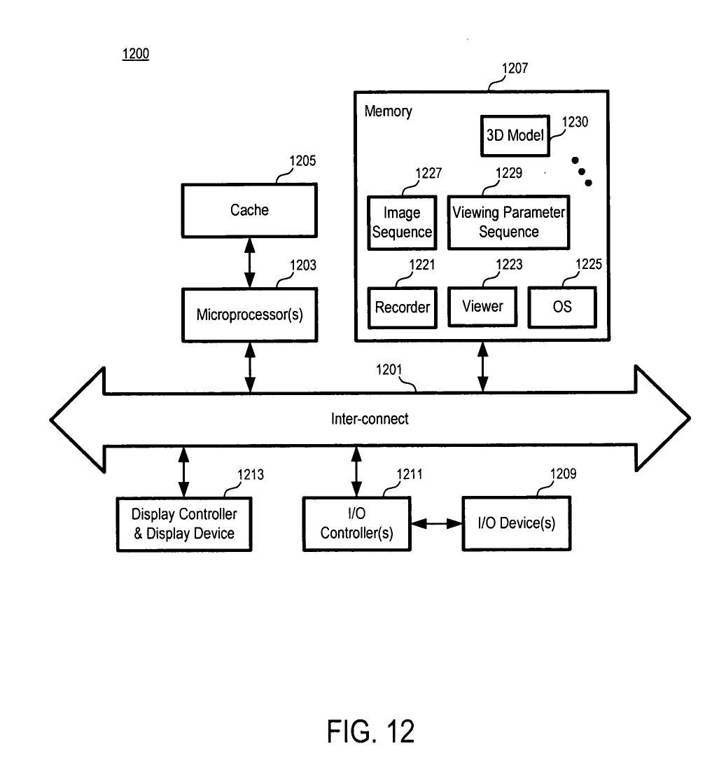

FIG. 12 shows a block diagram example of a data processing system for recording and/or reviewing an image guided procedure with augmented reality according to one embodiment of the present invention.

While FIG. 12 illustrates various components of a computer system, it is not intended to represent any particular architecture or manner of interconnecting the components. Other systems that have fewer or more components may also be used with the present invention.

In FIG. 12, the computer system (1200) is a form of a data processing system. The system (1200) includes an inter-connect (1201) (e.g., bus and system core logic), which interconnects a microprocessor(s) (1203) and memory (1207). The microprocessor (1203) is coupled to cache memory (1205), which may be implemented on a same chip as the microprocessor (1203).

The inter-connect (1201) interconnects the microprocessor(s) (1203) and the memory (1207) together and also interconnects them to a display controller and display device (1213) and to peripheral devices such as input/output (I/O) devices (1209) through an input/output controller(s) (1211). Typical I/O devices include mice, keyboards, modems, network interfaces, printers, scanners, video cameras and other devices.

The inter-connect (1201) may include one or more buses connected to one another through various bridges, controllers and/or adapters. In one embodiment the I/O controller (1211) includes a USB (Universal Serial Bus) adapter for controlling USB peripherals, and/or an IEEE-1394 bus adapter for controlling IEEE-1394 peripherals. The inter-connect (1201) may include a network connection.

The memory (1207) may include ROM (Read Only Memory), and volatile RAM (Random Access Memory) and non-volatile memory, such as hard drive, flash memory, etc.

Volatile RAM is typically implemented as dynamic RAM (DRAM) which requires power continually in order to refresh or maintain the data in the memory. Non-volatile memory is typically a magnetic hard drive, flash memory, a magnetic optical drive, or an optical drive (e.g., a DVD RAM), or other type of memory system which maintains data even after power is removed from the system. The non-volatile memory may also be a random access memory.

The non-volatile memory can be a local device coupled directly to the rest of the components in the data processing system. A non-volatile memory that is remote from the system, such as a network storage device coupled to the data processing system through a network interface such as a modem or Ethernet interface, can also be used.

The memory (1207) may stores an operating system (1125), a recorder (1221) and a viewer (1223) for recording, rebuilding and reviewing the image sequence for an image guided procedure. Part of the recorder and/or the viewer may be implemented using hardware circuitry for improved performance. The memory (1207) may include a 3D model (1230) for the generation of virtual images. The 3D model (1230) used for rebuilding the image sequence in the viewer (1223) may be the same as the one used to provide the display during the image guided procedure. The 3D model may include volumetric image data. The memory (1207) may further store the image sequence (1227) of the real world images captured in real time during the image guided procedure and the viewing parameters sequences (including positions and orientations of the camera) 1229) for generating the virtual images based on the 3D model (1230) and for combining the virtual images with the recorded image sequence (1227) in viewer (1223).

Embodiments of the present invention can be implemented using hardware, programs of instruction, or combinations of hardware and programs of instructions.

In general, routines executed to implement the embodiments of the invention may be implemented as part of an operating system or a specific application, component, program, object, module or sequence of instructions referred to as “computer programs.” The computer programs typically comprise one or more instructions set at various times in various memory and storage devices in a computer, and that, when read and executed by one or more processors in a computer, cause the computer to perform operations necessary to execute elements involving the various aspects of the invention.

While some embodiments of the invention have been described in the context of fully functioning computers and computer systems, those skilled in the art will appreciate that various embodiments of the invention are capable of being distributed as a program product in a variety of forms and are capable of being applied regardless of the particular type of machine or computer-readable media used to actually effect the distribution.

Examples of computer-readable media include but are not limited to recordable and non-recordable type media such as volatile and non-volatile memory devices, read only memory (ROM), random access memory (RAM), flash memory devices, floppy and other removable disks, magnetic disk storage media, optical storage media (e.g., Compact Disk Read-Only Memory (CD ROMS), Digital Versatile Disks, (DVDs), etc.), among others. The instructions may be embodied in digital and analog communication links for electrical, optical, acoustical or other forms of propagated signals, such as carrier waves, infrared signals, digital signals, etc.

A machine readable medium can be used to store software and data which when executed by a data processing system causes the system to perform various methods of the present invention. The executable software and data may be stored in various places including for example ROM, volatile RAM, non-volatile memory and/or cache. Portions of this software and/or data may be stored in any one of these storage devices.

In general, a machine readable medium includes any mechanism that provides (i.e., stores and/or transmits) information in a form accessible by a machine (e.g., a computer, network device, personal digital assistant, manufacturing tool, any device with a set of one or more processors, etc.).

Aspects of the present invention may be embodied, at least in part, in software. That is, the techniques may be carried out in a computer system or other data processing system in response to its processor, such as a microprocessor, executing sequences of instructions contained in a memory, such as ROM, volatile RAM, non-volatile memory, cache or a remote storage device.

In various embodiments, hardwired circuitry may be used in combination with software instructions to implement the present invention. Thus, the techniques are not limited to any specific combination of hardware circuitry and software nor to any particular source for the instructions executed by the data processing system.

In this description, various functions and operations are described as being performed by or caused by software code to simplify description. However, those skilled in the art will recognize what is meant by such expressions is that the functions result from execution of the code by a processor, such as a microprocessor.

Although some of the drawings illustrate a number of operations in a particular order, operations which are not order dependent may be reordered and other operations may be combined or broken out. While some reordering or other groupings are specifically mentioned, others will be apparent to those of ordinary skill in the art and so do not present an exhaustive list of alternatives. Moreover, it should be recognized that the stages could be implemented in hardware, firmware, software or any combination thereof.

In the foregoing specification, the invention has been described with reference to specific exemplary embodiments thereof. It will be evident that various modifications may be made thereto without departing from the broader spirit and scope of the invention as set forth in the following claims. The specification and drawings are, accordingly, to be regarded in an illustrative sense rather than a restrictive sense.

What is claimed is:

1. A method, comprising:
recording a sequence of positional data of a navigation instrument relative to a patient during a surgical navigation process.

2. The method of claim 1, wherein the positional data is the position of the navigation instrument.

3. The method of claim 1, wherein the positional data is the position and orientation of the navigation instrument; the position and orientation represents a viewpoint of the navigation instrument.

4. The method of claim 1, wherein the navigation instrument is tracked by a position tracking system and the positional data is generated at least in part from the tracking data.

5. The method of claim 1, further comprising:
generating at least one image of preoperative image data of the patient, the generated image is relative to the positional data.

6. The method of claim 1, wherein the navigation instrument comprises an imaging device.

7. The method of claim 6, further comprising:
overlaying the generated image and an image obtained from the imaging device.

8. The method of claim 7, further comprising:
recording a sequence of images obtained from the imaging device in association with the sequence of positional data.

9. The method of claim 6, wherein the imaging device is one of: a video camera, an endoscope, a microscope, or an ultrasound probe.

10. The method of claim 1, wherein said recording is started and/or ended automatically based on a predefined condition.

11. The method of claim 1, wherein said recording is started and/or ended automatically based on a user input,

12. The method of claim 1, wherein the navigation instrument is a probe with a video camera affixed to the probe.

13. A method of claim 12, comprising:
tracking positions and orientations of the probe during a surgical navigation process; and
recording the positions and orientations of the probe, the recording of the positions and orientations to be used to subsequently generate images based on preoperative images of a patient.

14. The method of claim 13, further comprising:
recording video images from the camera of the probe during the navigation, in association with the positions and orientations of the probe.

15. The method of claim 14, further comprising:
generating images in real-time for navigation during the recording; and
mixing the generated images with corresponding video images in real-time for navigation during the recording.

16. The method of claim 13, further comprising:
recording a frame of video from the camera; and
separately recording the position and orientation of the camera in association with the frame of the video.

17. The method of claim 15, wherein said generating comprises:
generating the sequence of views for navigation based at least partially on user input.

18. A method as in claim 17, further comprising recording user input variables in generating the navigation view of three dimensional image data.

19. A method as in claim 18, wherein the recording the user input variables comprises recording the user input variables separate from the recording of the video, and synchronized with at least one of the recording of the positions and orientations of the camera, or the recording of the video.

20. A method as in claim 18, wherein the user input variables comprises one or more of changes in transparency, visibility, lighting, color, zooming in, and zooming out.

21. A method as in claim 18, further comprising recording one or more navigational events searchable during navigation review.

22. A method as in claim 21, wherein the recording one or more navigational events comprises recording the navigational events separate from the recording of the video, and synchronized with at least one of the recording of the positions and orientations of the camera, or the recording of the video.

23. A method as in claim 22, wherein the navigational events comprise changes in visibility in one or more of tumors, blood vessels, nerves, a surgical path, or a pre-identified anatomical landmarks.

24. A method as in claim 18, further comprising recording verbal commentary of a user, wherein the recording of the verbal commentary is synchronized with one of the recording of the positions and orientations of the camera, or the recording of the video.

25. A method implemented on a data processing system, the method comprising:
reading a recorded data set of a navigation process, said data set is recorded with one of the recording methods disclosed in this invention;
re-generating a sequence of views of the navigation process based on the recorded data set.

26. A method as in claim 25, further comprising retrieving, subsequent to the surgical procedure, the positional data from the recorded data set to re-generate views of the three dimensional image data for reviewing the navigation process.

27. A method as in claim 25, further comprising recording the re-generated sequence of views of navigation process as a video.

28. A method as in claim 25, further comprising retrieving, subsequent to the surgical procedure, the positions and orientations of the camera from the recorded data set to re-generate views of the three dimensional image data for reviewing the navigation process.

29. A method as in claim 28, further comprising overlaying the views of the three-dimensional image data over a playback of the recorded video retrieved from the recorded data set.

30. A method as in claim 29, wherein the three-dimensional image data have been updated after the recording of the navigation process.

31. A method as in claim 26, further comprising modifying the views of the three-dimensional image data during reviewing of the navigation process.

32. A method as in claim 31, wherein the modifying comprises modifying at least one of lighting, color, transparency, magnification or visibility of a portion of the three-dimensional image data, or changing one or more models of anatomical structures in the three-dimensional image data.

33. A method for transmitting navigation process over a network, comprising: transmitting at least the positional data of the navigation instrument over a network.

34. A method as in claim 33, wherein the navigation instrument is a probe with a video camera affixed to the probe.

35. A method as in claim 34, comprising: transmitting at least the positional data and the video image of the video camera over a network.

36. A method as in claim 33, wherein the position data is recorded using a recording method as disclosed in this invention.

37. A method as in claim 35, wherein the position data and the video image are recorded using a recording method as disclosed in this invention.

38. A method as in claim 37, further comprising transmitting over a network, in accordance with available bandwidth, at least one of recorded positions and orientations of the camera or the recorded video.

39. A method as in claim 38, wherein the recorded positions and orientations of the camera and the recorded video are to be transmitted to display remotely from the surgical procedure, views of the three-dimensional image data, overlaid with and in synchronization with a playback of the recorded video.

40. A machine readable media embodying instructions, the instructions causing a machine to perform a method, the method comprising:
recording a sequence of positional data to represent a position or position and orientation of a navigation instrument relative to a patient during a surgical navigation process.

41. A data processing system, comprising:
means for receiving a sequence of positional data to represent a position or position and orientation of a navigation instrument relative to a patient during a surgical navigation process; and
means for recording the sequence of the positional data during the surgical navigation process.

42. A data processing system, comprising:
memory; and
one or more processors coupled to the memory, the one or more processors to record in the memory a sequence of positional data to represent a position or position and orientation of a navigation instrument relative to a patient during a surgical navigation process.

43. A machine readable media embodying data recorded from executing instructions, the instructions causing a machine to perform a method, the method comprising:
during a surgical procedure of an object, recording a sequence of positional data to represent a position or position and orientation of a navigation instrument relative to a patient during a surgical navigation process.

44. A system, comprising:
a position tracking system to generate tracking data of a navigation instrument during a surgical navigation process; and
a computer coupled to the position tracking system, during a surgical procedure of an object, the computer to record a sequence of positional data to represent a position or position and orientation of a navigation instrument relative to a patient during a surgical navigation process, based at least partially on the tracking data.
